(19) United States
(12) Patent Application Publication
(10) Pub. No.: US 2008/0045967 A1
(43) Pub. Date: Feb. 21, 2008

(54) Surgical Lever

(75) Inventors: Philip Lubinus, Kiel (DE); Arnold Keller, Hamburg (DE)

Correspondence Address: MORRISON & FOERSTER LLP, 1650 TYSONS BOULEVARD, SUITE 400, MCLEAN, VA, 22102, US

(73) Assignee: Waldemar Link GmbH & Co. KG, Hamburg (DE)

(21) Appl. No.: 11/630,266

(22) PCT Filed: Jun. 22, 2004
(86) PCT No.: PCT/EP04/06742

§ 371 Date: Oct. 16, 2007

(51) Int. Cl. A61B 17/58

(52) U.S. Cl. 606/90; 606/81

(57) ABSTRACT

Surgical lever, particularly for lifting the femur from the hip bone. It comprises an actuating handle (5) and an engagement device (6) and is characterized in that a projection (9, 11) dimensioned for support within the depth of the acetabulum is arranged between these two parts (5, 6). For adaptation to the inner surface of the acetabulum, said projection (9, 11) is expediently of a spherical configuration and can be made available with a varying radius.

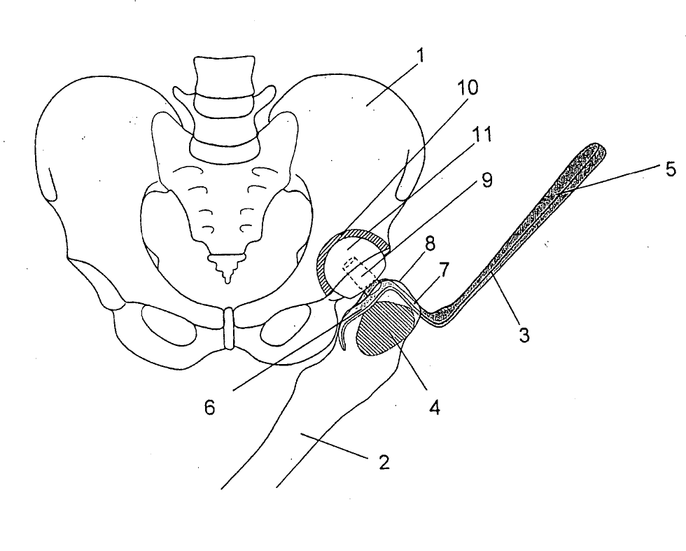

The invention relates to a surgical lever, particularly for lifting the femur from the hip bone.

Surgical levers of this kind are typically used in operations in which the natural hip joint is replaced by an artificial one, so that, after resection of the femoral head, the femur can be held in such a way that the femoral medullary cavity can be worked. If the resection is performed with the hip joint in the dislocated state, there is also the possibility of lifting the femur from the hip bone during the resection.

In the known surgical levers with which the femur is lifted from the hip bone, the margin of the acetabulum forms the abutment on which the surgical lever is supported. This is disadvantageous because, on the one hand, structures on the margin of the acetabulum may be damaged, and, on the other hand, the margin of the acetabulum does not offer a secure abutment, for example one that avoids lateral slipping of the lever.

The object of the invention is to make available a surgical lever that avoids these disadvantages.

The object is achieved by the fact that a projection dimensioned for support within the depth of the acetabulum is arranged between the actuating handle and the engagement device. Because of its concave shape, the depth of the acetabulum offers a stable abutment for the surgical lever. By virtue of the concavity of the acetabulum, the projection, once in position, obtains a secure hold therein.

A further advantage offered by the acetabulum, by virtue of its concave shape, is that the direction in which the abutment acts against the surgical lever is not defined in a fixed manner. The direction in which the lever is supported in the acetabulum can differ depending on the particular situation. The support within the depth of the acetabulum affords safety under any direction of force. The femur can be lifted from the hip bone in different directions.

In an advantageous embodiment, the projection of the surgical lever has a support surface which has a convex configuration matching the curvature of the acetabulum. Local loading of the acetabulum is avoided by means of this configuration, since the support surface acts on a larger surface area.

It is also advantageous if several support surfaces with different radii of curvature are made available and are used alternately depending on the size or radius of curvature of the acetabulum. The acetabula of different people are differently shaped and, in particular, have different diameters. By means of the support surfaces with different radii of curvature, the surgical lever can be used on the greatest possible number of people. Several levers with differently curved support surfaces are provided, or a number of exchangeable parts forming differently curved support surfaces are provided.

In an advantageous embodiment, the support surface has an at least hemispherical configuration. This makes it particularly easy to insert into the acetabulum, and it automatically centers itself therein.

The connection between surgical lever and support surface is preferably established via a conical plug connection, the cone angle being chosen such that the support surface on the one hand does not inadvertently come loose and on the other hand can be exchanged for another one by simple removal and replacement. The part forming the support surface can be connected rigidly to the lever. During lever movement, the support surface then slides within the acetabulum. It is also possible, however, to join this part to the lever in an articulated manner, such that the lever movement takes place at the hinge, while the support surface remains stationary in the acetabulum.

The engagement device advantageously has a concave shape in the working direction, in such a way that it can safely engage the external shape of the bone, particularly of the trochanter. The concave configuration is suitable to avoid the trochanter slipping from the surgical lever.

The invention is described below with reference to the attached drawing and on the basis of an advantageous illustrative embodiment. The single FIGURE shows a plan view of a surgical lever according to the invention in use on a hip joint.

A human hip bone is shown, and also part of the left femur 2, of which the femoral head has been resected in the area of the neck of the femur and removed. The resection surface 4 and the acetabulum 10 are exposed. The femur 2 is lifted from the hip bone 1 with the aid of a surgical lever 3 according to the invention and is inwardly rotated in such a way that the femoral medullary cavity is accessible for working by the operating surgeon. The surgical lever 3 engages on the greater trochanter 8 of the femur 2.

The surgical lever 3 comprises an actuating handle 5 and an engagement device 6. The actuating handle 5 and the engagement device 6 are elongate elements which, at their inner ends, are rigidly connected via an intermediate piece 7 that extends substantially perpendicular to both of them. At its end, the engagement device 6 is shaped concavely by formation of a curved-back nose and is thereby adapted to the external shape of the greater trochanter 8.

A cone 9 is arranged at the inner end of the engagement device 6. The cone 9, set back slightly in the direction of the outer end of the engagement device 6, forms a continuation of the intermediate piece 7. A sphere segment 11 is connected to the cone 9 via a conical plug connection, the sectional surface of said sphere segment 11 being provided with a conical bore that matches the cone 9. The outer surface of the sphere segment 11 is located within the acetabulum 10 and is shaped, as a support surface, according to the curvature of the acetabulum 10. The cone 9, together with the sphere segment 11, forms a projection via which the surgical lever 3 is supported within the depth of the acetabulum 10.

The sphere segment 11 is held securely by the acetabulum 10. The sphere segment 11 is not moved from its position, even by forces acting transverse to the cone 9. Moreover, the sphere segment 11 can be turned in all directions within the acetabulum 10, such that the surgical lever can be applied from different directions and also at different angles. By way of the sphere segment 11, the acetabulum 10 forms an abutment that permits safe and versatile use of the surgical lever 3.

1. A surgical lever, particularly for lifting the femur (2) from the hip bone (1), comprising an actuating handle (5) and an engagement device (6), characterized in that a projection (9, 11) dimensioned for support within the depth of the acetabulum is arranged between the actuating handle (5) and the engagement device (6).

2. The surgical lever as claimed in claim 1, characterized in that the projection has a convex support surface (11).

3. The surgical lever as claimed in claim 1 or 2, characterized in that the lever can be made available with support surfaces (11) having a varying radius of curvature.

4. The surgical lever as claimed in one of claims 1 through 3, characterized in that a part (11) forming the support surface is exchangeable and can be made available with a varying radius of curvature.

5. The surgical lever as claimed in one of claims 1 through 4, characterized in that the support surface (11) has an at least hemispherical configuration.

6. The surgical lever as claimed in claim 5, characterized in that the connection between surgical lever and support surface (11) is a conical plug connection.

7. The surgical lever as claimed in one of claims 1 through 6, characterized in that the engagement device (6) has a concave configuration in the working direction.

1. A surgical lever, particularly for lifting the femur (2) from the hip bone (1), with an actuating handle (5), an engagement device (6), and a projection (9, 11) dimensioned for support within the depth of the acetabulum and arranged between the actuating handle (5) and the engagement device (6), the projection having a convex support surface (11), characterized in that the support surface (11) is connected to the surgical lever via a conical plug connection.

2. The surgical lever as claimed in claim 1, characterized in that the lever can be made available with support surfaces (11) of varying radius of curvature.

3. The surgical lever as claimed in claim 1 or 2, characterized in that a part (11) forming the support surface is exchangeable and can be made available with a varying radius of curvature.

4. The surgical lever as claimed in one of claims 1 through 3, characterized in that the support surface has an at least hemispherical configuration.

5. The surgical lever as claimed in one of claims 1 through 4, characterized in that the engagement device (6) has a concave configuration in the working direction.
